(19) United States
(12) Patent Application Publication
(10) Pub. No.: US 2026/0074066 A1
(43) Pub. Date: Mar. 12, 2026

(54) Establishing Wireless Data Connections Between Medical Devices

(71) Applicant: Stryker Corporation, Portage, MI (US)

(72) Inventor: Justin Michael Andrews, Schoolcraft, MI (US)

(73) Assignee: Stryker Corporation, Portage, MI (US)

(21) Appl. No.: 19/100,358

(22) PCT Filed: Aug. 2, 2023
(86) PCT No.: PCT/US2023/029305

§ 371 Date: Jan. 31, 2025

Related U.S. Application Data
(60) Provisional application No. 63/370,124, filed on Aug. 2, 2022.

(51) Int. Cl. G16H 40/67; A61M 1/00; G06K 7/10; G06K 7/14

(52) U.S. Cl. CPC G16H 40/67; A61M 1/60; G06K 7/10297; G06K 7/1408; A61M 2205/3379; A61M 2205/3592; A61M 2205/502; A61M 2205/6072; A61M 2230/20

(57) ABSTRACT

Systems, methods, and computer program products for establishing wireless connections between user interface devices and surgical devices. A medical system includes a surgical device remote from a user interface device. A wireless data connection between the surgical and the user interface device is facilitated by a cartridge forming a functional component of the surgical device during operation of the surgical device to perform a surgical task. The cartridge includes a tag having data stored thereon. The tag is read with the user interface device and the surgical device, and the surgical device and the user interface device are paired to establish a wireless connection based on the data read from the tag.

### RELATED APPLICATIONS

This application claims priority to and all the benefits of United States Provisional Application No. 63/370124, filed Aug. 2, 2022, the entire contents of which is hereby incorporated by reference.

### BACKGROUND

Surgical procedures frequently involve the use of medical systems that incorporate computer controllers. Such medical systems often include several devices, such as data-producing devices, data-consuming devices, and control systems, in communication with one another to facilitate operation of the systems. Reliable data communication between the multiple devices helps to ensure proper functioning of the systems. It would be advantageous to provide medical systems with intuitive pairing processes to facilitate wireless data communication between devices that overcome the drawbacks and deficiencies present in previously known medical systems.

### SUMMARY

A system for use within an operating suite can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by a data processing apparatus, cause the apparatus to perform the actions. In one aspect, a method of establishing a wireless connection within a system between a user interface device and a surgical device via a cartridge that includes a tag for storing a unique identifier is provided. The surgical device can include a first reader, and a first communication device. The user interface device can include a second reader and a second wireless communication device.

The method can include reading, with the second reader, the unique identifier of the tag of the cartridge positioned in proximity to the second reader. The surgical device can be configured to establish one or more of a mechanical, electrical, or fluid coupling with the cartridge. The cartridge can be positioned in proximity to the first reader upon the coupling being established. The cartridge can comprise a functional component of the surgical device during operation. The method can include reading, with the first reader, the unique identifier of the tag of the cartridge positioned in proximity to the first reader. The method can include broadcasting, with the first wireless communication device, the unique identifier and a wireless device address. The method can include scanning, with the second wireless communication device, for the unique identifier. The method can include pairing the surgical device and the user interface device via the wireless device address based on the scanned unique identifier matching the broadcasted unique identifier.

In some implementations, the method can include generating a wireless device name including the unique identifier and scanning, with the second wireless communication device, for the wireless device name. In some implementations, the surgical device can include a receiver, and the method can include removably receiving the cartridge within the receiver to establish a mechanical coupling between the surgical device and the cartridge. In some implementations, the method can include detecting an insertion of the cartridge within the receiver and activating the first reader based on the detected insertion.

In some implementations, the surgical device can include a vacuum source in fluid communication with the receiver, and the cartridge can be a manifold configured to be removably coupled with a suction tube. The method can include operating the vacuum source of the surgical device to draw surgical waste through the suction tube and the manifold such that the manifold facilitates the wireless connection and is a functional component of the surgical device during operation.

In some implementations, the surgical device can include a waste container in fluid communication with the receiver and a fluid measuring system operably coupled to the waste container. The method may include determining, with the fluid measuring system, a fluid volume within the waste container. The method may also include transmitting fluid volume data from the surgical system to the user interface device with the wireless connection. The method may also include displaying, with the user interface device, the fluid volume data.

In some implementations, the surgical device and/or the cartridge can include means for determining a concentration of blood within the surgical waste. The method can include determining the concentration of blood within the surgical waste. The method can include determining a blood volume within the waste container based on the determined concentration of blood and the determined fluid volume. The method can include transmitting blood volume data from the surgical device to the user interface device via the wireless connection. The method can include displaying, with the user interface device, the blood volume data.

In some implementations, the surgical device can further include a waste container and a vacuum source in fluid communication with the waste container. The surgical device and the cartridge can include means for determining a concentration of blood within the surgical waste. The method can include operating the vacuum source of the surgical device to draw surgical waste through the cartridge. The method can also include determining the concentration of blood within the surgical waste being drawn through the cartridge. The method can further include determining a flow rate of the surgical waste being drawn through the cartridge. The method can further include determining a blood volume within the waste container based on the determined concentration of blood and the determined flow rate. The method can additionally include transmitting the blood volume data from the surgical device to the user interface device via the wireless connection. The method can also include displaying, with the user interface device, the blood volume data.

In some implementations, the tag comprises an RFID tag, and the method can include writing or rewriting data to the tag. The data written or rewritten to the tag may prevent reuse or reprocessing of the cartridge.

In some implementations, the first reader and second readers are optical sensors. The tag may include a machine-readable optical code. The wireless connection may be established by the Bluetooth wireless pairing protocol.

In another aspect, a method of establishing a wireless connection between a surgical device and a user interface device via a cartridge is provided. The cartridge can include a tag storing a unique identifier. The surgical device can include a vacuum source, a first reader and a first wireless communication device. The user interface device can include a second reader and a second wireless communication device. The method can include positioning the cartridge in proximity to the second reader. The method can include confirming, by output displayed on the user interface device, the unique identifier of the tag has been read by the second reader. The cartridge may be coupled with the surgical device to position the cartridge in proximity to the first reader. The method can include reading the unique identifier of the tag with the first reader. The method can include establishing the wireless connection between the surgical device and the user interface device based on the unique identifier being scanned by the second wireless communication device matching the unique identifier being broadcast by the first wireless communication device. The method can include operating the vacuum source to draw surgical waste through the cartridge. The method can include viewing, on the user interface device, information transmitted to the user interface device from the surgical device via the wireless connection.

In some implementations, the surgical device includes a receiver, and the cartridge may be coupled to the surgical device by being inserted into the receiver. Inserting the cartridge into the receiver can establish one or more of a mechanical, fluid, or electrical connection with the surgical device, and can position the cartridge in proximity to the first reader.

In some implementations, the method can include confirming, by output displayed on the user interface device, the unique identifier of the tag has been read by the first reader. In some implementations, the method can include confirming, by output displayed on the user interface device, that the wireless connection has been established.

In some implementations, the surgical device can be positioned within an operating suite at a first location, and the user interface device can be positioned within the operating suite at a second location relative to the first location that precludes use of near field communication to establish the wireless connection.

In other aspects, computer systems, apparatuses, and computer program products including computer-executable instructions recorded on one or more computer storage devices are provided, each configured to perform the actions of one or more of the above-described methods.

In a further aspect, a system for use within an operating suite is provided. The system can include a cartridge that includes a tag storing a unique identifier. The system can include a surgical device including a vacuum source. The surgical device can include a waste container in fluid communication with the vacuum source. The surgical device can include a receiver in fluid communication with the waste container. The surgical device can include a first reader coupled to the receiver. The surgical device can include a first wireless communication device. The receiver can be configured to removably receive the cartridge to position the tag in proximity with the first reader. The system can include a user interface device positionable remote from the surgical device. The user interface device can include a display. The user interface device can include a second reader. The user interface device can include a second wireless communication device in communication with the second reader. The first wireless communication device can be configured to broadcast the unique identifier and the device address based on a reading of the tag by the first reader. The second wireless communication device can be configured to scan for the unique identifier based on a reading of the tag by the second reader so as to pair and establish a wireless connection between the surgical device and the user interface device via the wireless device address.

In an additional aspect, a system for establishing a wireless connection between devices in a medical setting is provided. The system can include a cartridge including a tag storing a unique identifier is provided. The system can include a surgical device including a vacuum source. The surgical device can include a waste container in fluid communication with the vacuum source. The surgical device can include a first reader. The surgical device can include a first wireless communication device. The cartridge can be configured to be arranged in fluid communication with the vacuum source at a position in which the tag is in proximity with the first reader. The system can include a user interface device positionable remote from the surgical device. The user interface device can include a display. The user interface device can include a second reader. The user interface device can include a second wireless communication device in communication with the second reader. The first wireless communication device can be configured to broadcast the unique identifier and the device address based on a reading of the tag by the first reader. The second wireless communication device can be configured to scan for the unique identifier based on a reading of the tag by the second reader so as to pair and establish a wireless connection between the surgical device and the user interface device via the wireless device address.

In some implementations of the above systems, the surgical device can include a means for determining blood volume data as a measure of blood within surgical waste being drawn under the influence of vacuum from the vacuum source. The surgical device can be configured to transmit the blood volume data across the wireless connection. The user interface device can be configured to display the blood volume data on the display of the user interface device.

In some implementations of the above systems, the first reader and the second reader can be optical sensors, and the tag can include a machine-readable optical code.

In some implementations of the above systems, the first reader can include one of an optical sensor and an RFID interrogator, the second reader can include the other of the optical sensor and the RFID interrogator, and the tag can include a machine-readable optical code and an RFID tag. In some implementations, the first reader includes the RFID interrogator, and the second reader includes the optical sensor. In some implementations, the first reader can not include the other of the optical sensor and the RFID interrogator, the second reader can not include the one of the optical sensor and the RFID interrogator, and the machine-readable optical code and the RFID tag can each indicate the unique identifier.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 illustrates a medical system for use in an operating suite according to the present disclosure, the system including a user interface device, a surgical device, and a cartridge configured to be removably received by the surgical device and to facilitate pairing between the user interface device and the surgical device.

FIG. 2 illustrates an example in accordance with the system of FIG. 1.

FIG. 3 is an enlarged view of the cartridge of FIG. 2.

FIG. 4 illustrates another example in accordance with the system of FIG. 1.

FIG. 5 illustrates a further example in accordance with the system of FIG. 1

FIG. 6 illustrates an alternative view of the cartridge of FIG. 5.

FIG. 7 illustrates a flowchart of a method for establishing a wireless connection between a surgical device and a user interface device in accordance with the present disclosure.

### DETAILED DESCRIPTION

Aspects of the present disclosure generally relate to an intuitive and improved pairing process for facilitating wireless data communication between devices of a surgical environment. A given surgical procedure may involve several surgical devices each performing a different surgical task to assist in the procedure. For instance, one exemplary surgical procedure may utilize an ultrasonic tool device for treating tissue at a surgical site, a medical waste collection device for collecting medical waste from the target site as a result of treatment, and a surgical sponge management device for tracking surgical sponges used during the procedure. Each of these devices may be separately positionable within the operating suite, and may include a local user interface for providing operational information and accepting user input for the device. Such a configuration is deficient as a means assisting in a surgical procedure, however, as it can be inconvenient or impractical in some cases for surgical personnel to continuously digest data from and provide user input to the local user interface of each device while also attending to the patient during a surgical procedure.

Accordingly, the present disclosure describes systems, methods and computer program products that may function to consolidate the data output and user input functions of multiple surgical devices at a single point of access, namely, a user interface device, which may be easily repositioned during a procedure at the surgical personnel's convenience. One option to accomplish this goal may be to integrate the multiple surgical devices into a single unit or connect the devices by wires or cables to carry the data therebetween. This configuration, however, has the drawback of limiting the ability of the devices to separate and/or move relative to each other at the convenience of surgical personnel during a procedure, and also crowds the surgical environment. The present description thus provides systems, methods, and computer products for establishing a wireless communication between surgical devices to help overcome the drawbacks of wired systems, and does so in a way that avoids the potential downfalls of a wireless configuration, such as complex pairing processes, the lack of flexibility in swapping different surgical devices between multiple surgical environments, and the limited range of proximity-based communication technologies such as near-field communication (NFC).

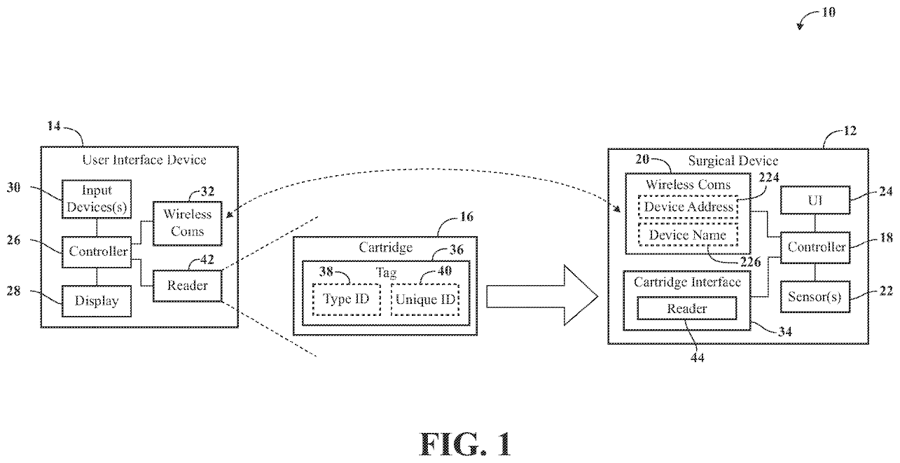

FIG. 1 illustrates a surgical system 10 including a surgical device 12, a user interface device 14, and a cartridge 16. The cartridge 16 may include or form a functional component of the surgical device 12 that cooperates with the surgical device 12 to perform a surgical task related to a surgical procedure. In some instances, the cartridge 16 may be configured to be removably received by the surgical device 12 to enable performance of the surgical task. The cartridge 16 may also be configured to facilitate establishing a reliable wireless connection between the surgical device 12 and user interface device 14 in an intuitive manner. For instance, the wireless connection may be established by virtue of the cartridge 16 being disposed in its operative position for cooperating with the surgical device 12 to perform a surgical task, thus limiting the number of steps needing to be performed by surgical personnel to establish the connection when preparing the system 10 for a procedure. In some instances, the cartridge 16 may be a disposable, single use component configured to cooperate with the surgical device 12 to perform the surgical task.

Once established, the wireless connection may enable the surgical device 12 to communicate data to the user interface device 14 for display to surgical personnel. The wireless connection may also enable the user interface device 14 to receive user input commands for the surgical device 12, and to communicate those commands to the surgical device 12 for execution. In certain implementations, the surgical device 12 may also communicate with remote devices, such as devices accessible through the Internet, through the user interface device 14.

The surgical device 12 may be configured to perform at least one surgical task in relation to a surgical procedure. Non-limiting examples of such a surgical task may include treating tissue at a surgical site, suctioning medical waste from the surgical site as a result of the procedure, and tracking sponges used in the procedure so as to avoid accidental retention at the surgical site.

The surgical device 12 may include a controller 18 configured to control operation of the surgical device 12, including implementation of the functions, features, and processes of the surgical device 12 described herein. The surgical device 12 may also include a wireless communication device 20 in communication with the controller 18. The wireless communication device 20 may be configured to provide wireless data communications between the surgical device 12 and other devices and systems, such as via radio frequency signals. The wireless communication device 20 may incorporate relatively short-range communications technology, such as Bluetooth. In some examples, the wireless communication device 20 may lack relatively long-range communications capabilities, such as Wi-Fi and cellular.

The surgical device 12 may also include one or more sensors 22 in communication with the controller 18. The sensor(s) 22 may be configured to generate operational data indicative of use of the surgical device 12. The controller 18 may be configured to receive the operational data generated by the sensor(s) 22, and to regulate operation of the surgical device 12 based thereon. The controller 18 may also be configured to generate user data based on the operational data for output to surgical personnel.

In some instances, the surgical device 12 may also include a local user interface (UI) 24 in operable communication with the controller 18. The local UI 24 may include one or more output devices, such as one of more of a display and/or speaker, for presenting information, such as the operational data generated by the sensor(s) 22, from the controller 18 to the user. The local UI 24 may also include one or more input devices, such as a touchscreen display, mouse, keyboard, and/or microphone, for accepting user input. Surgical personnel may thus be able to enter commands to the controller 18 for regulating operation of the surgical device 12 via the local UI 24.

As previously described, the local UI 24 may be inconvenient or inadequate for the purposes of relaying information to surgical personnel relating to operation of the surgical device 12, and for receiving user input for regulating operation of the surgical device 12. Thus, according to aspects of the present disclosure, the surgical device 12 may be paired with a user interface device 14 so as to establish a wireless connection therebetween, which may enable the user interface device 14 to function as a remote UI of the surgical device 12. The user interface device 14 may then be positioned at the convenience of surgical personnel. In some instances, the user interface device 14 may also function as a surgical device configured to perform another surgical task in relation to the surgical procedure.

The user interface device 14 may include a controller 26 configured to control operation of the user interface device 14, including implementation of the functions, features, and processes of the user interface device 14 described herein. For instance, the controller 26 may be configured to control the operation of the surgical device 12, such as based on user input received for the surgical device 12, to receive and process operational data related to the operation of the surgical device 12, and to aggregate data from multiple surgical devices 12 paired with the user interface device 14. As one non-limiting example, the user interface device 14 may be paired with multiple blood loss measurement devices being utilized during a procedure, and the controller 26 may be configured to compile blood loss data from each of such devices to provide more accurate real-time analysis of blood loss during the surgical procedure.

The user interface device 14 may also include a display 28 and one or more input devices 30 each in electronic communication with the controller 26. The input device(s) 30 may include a touchscreen integrated with the display 28. The input device(s) 30 may also include a keyboard or keypad, a mouse or other pointing device, a microphone, or other input devices known in the art. The user interface device 14 may also include a wireless communication device 32 coupled to the controller 26. The wireless communication device 32 may complement the wireless communication device 20 of the surgical device 12 so as to enable establishing a wireless connection therebetween. For instance, the wireless communication device 32 may incorporate relatively short-range communications technology such as Bluetooth for communication with similar technology of the surgical device 12. The wireless communication device 32 may also include technology enabling relatively long-range communications capabilities, such as Wi-Fi and cellular.

The cartridge 16 may be configured to facilitate pairing the wireless communication devices 20, 32 to enable unidirectional or bidirectional communication between the surgical device 12 and the user interface device 14. The cartridge 16 may also form an operative component of the surgical device 12, and in this way may be configured to enable performance of a surgical task by the surgical device 12. The surgical device 12 may include a cartridge interface 34 for removably receiving the cartridge 16, such as to form one or more of a fluid, mechanical, or electrical coupling with the cartridge 16, which may facilitate performance of the surgical task.

The cartridge 16 may include a tag 36 containing data usable by the surgical device 12 and the user interface device 14 in the pairing process. For instance, the tag 36 may indicate one or more of a type identifier 38 or a unique identifier 40. The type identifier 38 may generally indicate the type of cartridge 16, and may be realized as a part or model number assigned to all cartridges 16 of the given type. The unique identifier 40 may vary across different cartridges 16 of the same type, and may be used by both the surgical device 12 and user interface device 14 to establish a wireless connection therebetween.

The surgical device 12 and the user interface device 14 may each include a means for reading the tag 36 data. For instance, the user interface device 14 may include a reader 42, such as an RFID or optical reader, for reading the tag 36 data when the cartridge 16 is brought in proximity to the reader 42. The surgical device 12 may include a corresponding reader 44. The reader 44 may be positioned relative to the cartridge interface 34 of the surgical device 12 such that, upon the cartridge 16 being coupled to the surgical device 12, the controller 18 may read the data from the tag 36 via the reader 44.

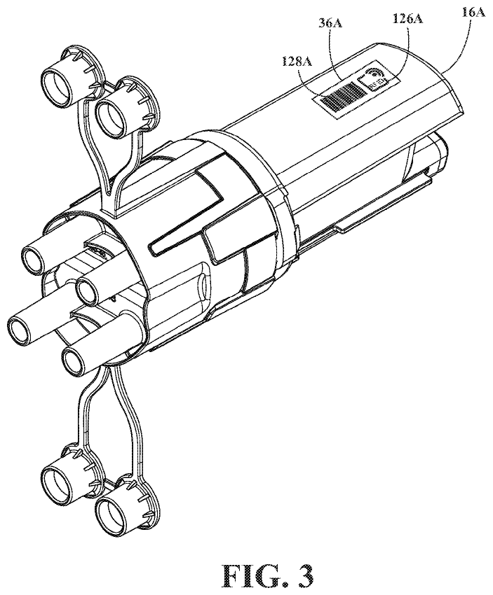

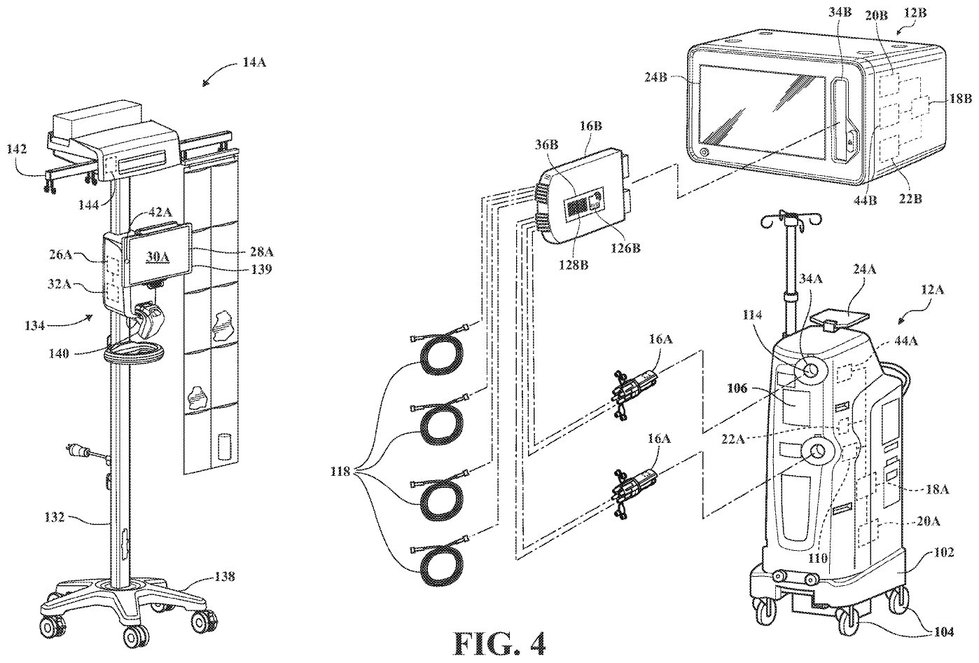

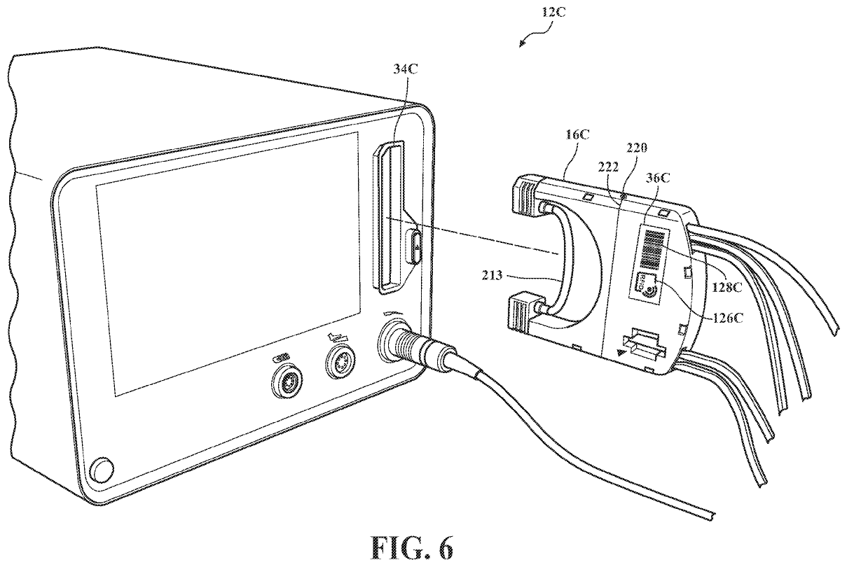

The tag 36 may include an RFID tag (e.g., RFID tag 126A (FIGS. 3), 126B (FIG. 4), and 126C (FIG. 6)), such as an RFID tag that complies with ISO/IEC 18000, ISO/IEC 15693, or ISO/IEC 14443 standards or other similar protocols. The tag 36 may also employ other radio-based communication protocols. At least one of the readers 42, 44 (e.g., both readers 42, 44) may include an RFID interrogator arranged to communicate on the same protocol as the tag 36. Additionally or alternatively, the tag 36 may include optically-encoded machine-readable data, such as a barcode (e.g., barcode 128A (FIGS. 3), 128B (FIG. 4), and 128C (FIG. 6)), or a QR-code. In this case, at least one of the readers 42, 44 (e.g., both readers 42, 44) may include an optical scanner arranged to scan the optical encoding of information. In one example, the tag 36 may include a combination of RFID and optical encoding, optionally with each storing the same data (e.g., type identifier 38, unique identifier 40), and the readers 42, 44 may be of different types, where the surgical device 12 employs one type of reader, RFID or optical, for reading the data from the tag 36, and the user interface device 14 employs the other type of reader for reading data from the tag 36. In a further example, the tag 36 may include a combination of RFID and optical encoding, optionally with each storing different data (e.g., the optical encoding storing the type identifier 38, and the RFID storing the unique identifier 40, or vice versa), and at least one of readers 42, 44 (e.g., both readers 42, 44) may include both an optical reader and an RFID reader for reading the data from the tag 36.

In some instances, the tag 36 may include a writable or rewritable data field where a reader 42, 44, such as an RFID interrogator of the reader 44, may be configured to write or rewrite data into the writable or rewritable data field to indicate usage of the cartridge 16 to which the tag 36 is applied. Once used to establish a wireless connection as described herein and/or to facilitate performance of a surgical task such as for a specified period as described herein, the cartridge 16 may be rendered inoperable to establish a further wireless connection between a surgical device 12 and a user interface device 14 and/or to facilitate further performance of a surgical task by virtue of the written or rewritten data. Should the cartridge 16 be thereafter used in an attempt to establish a wireless connection and/or facilitate performance of a surgical task as described herein, the information written to this field may cause the controller 18 and/or the controller 26 to determine that the cartridge 16 has been used and/or is unsuitable for further use in a surgical operation. Responsive to making such a determination, the controller 18 and/or the controller 26 may be configured to deny establishing a wireless connection and/or facilitating performance of a surgical task using the cartridge 16, and/or to provide a corresponding indicator to the user in the form of an alert or error message, such as on the local UI 24 and/or the display 28 of the user interface device 14.

Additionally or alternatively, responsive to a given cartridge 16 being used to establish a wireless connection as described herein and/or facilitate performance of a surgical task such as for a specified period as described herein, the controller 18 and/or the controller 26 may be configured to upload the data read from the tag 36 of the cartridge 16 to a used cartridge database. Such database may be maintained on one or more of the surgical device 12, the user interface device 14, or a remote server accessible over one or more networks such as the Internet. Should the cartridge 16 be thereafter used in connection with an attempt to establish a wireless connection and/or facilitate performance of a surgical task as described herein, the controller 18 and/or the controller 26 may be configured to cross check the data read from the tag 36 of the cartridge 16 against the used cartridge database to determine whether the tag 36 data is indicated by such database as used. If so, then the controller 18 and/or the controller 26 may be configured to deny establishing a wireless connection and/or facilitating performance of a surgical task using the cartridge 16, and/or to provide a corresponding indicator to the user in the form of an alert or error message, such as on the local UI 24 and/or the display 28 of the user interface device 14.

In some instances, rather than being incorporated into a cartridge 16 configured to be removably received by the surgical device 12, the tag 36 may be incorporated into a component that works in cooperation with but is configured to be disposed external to the surgical device 12 during operation. As an example, the tag 36 may be incorporated into a flow or other monitoring device that is external to but communicates information back to the surgical device 12, such as via a wired or relatively short-range wireless connection. In this case, the information provided on the tag 36 may still be utilized to facilitate the pairing of the surgical device 12 and user interface device 14, such as by positioning the component with the tag 36 in proximity to the reader 42, 44 of each device 12, 14 prior to disposing the component in an operative position to assist in the performance of the surgical task.

It is contemplated that a medical facility may possess multiple surgical devices 12 and and/or multiple user interface devices 14 that can be used in various configurations. Pairing a specific surgical device 12 to a specific user interface device 14 may enable proper communication between the devices in a particular operating suite. It is also possible that a single operating suite may utilize multiple surgical devices 12 and/or multiple user interface devices 14 for a single surgical operation. The pairing process allows surgical personnel to arrange and control the communication between specific pairs or groups of surgical devices 12 and user interface devices 14. The surgical device 12, the user interface device 14 and the pairing process are described in the following paragraphs in connection with various exemplary alternatives where similar features use similar reference numerals.

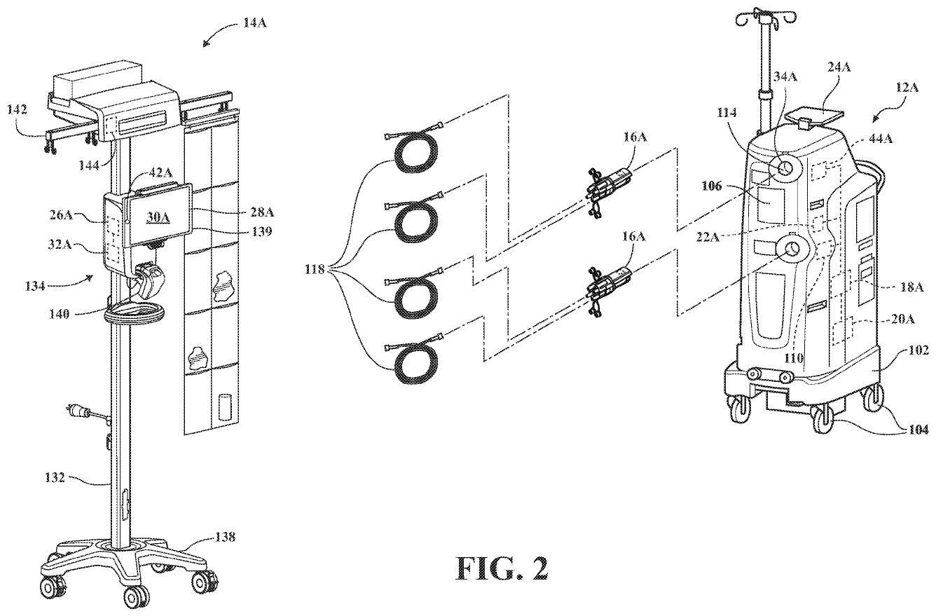

FIG. 2 illustrates an exemplary implementation in accordance with the surgical system 10 illustrated in FIG. 1. As shown in the illustrated example, a surgical device 12 may be realized as a waste collection unit 12A, and a user interface device 14 may be realized as a surgical sponge management device 14A.

The waste collection unit 12A may be configured to suction and collect the waste material from a surgical site, and may be configured to store the waste material until it is necessary or desired to off-load and dispose of the waste material. The waste collection unit 12A may include a base 102 and wheels 104 for moving the waste collection unit 12A along a floor surface within a medical facility. The waste collection unit 12A may also include at least one waste container 106 defining a waste volume for collecting and storing the waste material. The waste collection unit 12A may further include a vacuum source, or pump 110 (in phantom) supported on the base 102 and configured to draw suction on the waste container 106 through one or more vacuum lines. Suitable construction and operation of several subsystems of the waste collection unit 12A are disclosed in commonly owned United States Patent Publication No. 2005/0171495, published Aug. 4, 2005, International Publication No. WO 2007/070570, published Jun. 21, 2007, and International Publication No. WO 2014/066337, published May 1, 2014, the entire contents of each of which are hereby incorporated herein by reference.

The waste collection unit 12A may include at least one cartridge interface 34 realized as a receiver 34A supported on the base 102. Each of the receiver(s) 34A may define an opening 114 dimensioned to removably receive at least a portion of a cartridge 16, which as shown in the illustrated example may be realized as a manifold 16A. A suction path may be established from one or more suction tubes 118 to each waste container 106 through a manifold 16A when removably inserted into a receiver 34A. During a procedure, the vacuum generated by the vacuum pump 110 may be drawn on the suction tube(s) 118, and the waste material at the surgical site may be drawn through the manifold(s) 16A, through a suction outlet of the receiver(s) 34A, and into the waste container(s) 106.

The waste collection unit 12A may also include a controller 18A, which may correspond to the controller 18 described above in reference to FIG. 1. The controller 18A may provide for overall control of the waste collection unit 12A. For example, the controller 18A may be in communication with the vacuum pump 110, and may be configured to regulate the on/off operation of the vacuum pump 110. The controller 18A may also be configured to regulate the vacuum flow through the manifold(s) 16A. The controller 18A may further include or be in communication with a wireless communication device 20A of the waste collection unit 12A. The wireless communication device 20A may correspond to the wireless communication device 20 discussed above in reference to FIG. 1.

The waste collection unit 12A may also include one or more sensors 22A in communication with the controller 18A that are configured to generate operational data relating to use of the waste collection unit 12A. The sensor(s) 22A may correspond to the sensor(s) 22 described above in reference to FIG. 1. For example and without limitation, the sensor(s) 22A may include a fluid measuring subsystem arranged to measure the volume of waste material received in the waste container 106. Additionally or alternatively, the sensor(s) 22A may include one or more flow rate sensors to measure a rate of flow of material passing through the manifold 16A. Additionally or alternatively, the sensor(s) 22A may include one or more blood concentration sensors for measuring a concentration of blood within the waste material passing through the manifold 16A. Exemplary measurement or monitoring devices are disclosed in commonly-owned International Application No. PCT/US2021/058891, filed Nov. 11, 2021, the entire contents of which are hereby incorporated by reference. The data generated by the sensor(s) 22A may provide valuable information to surgical personnel regarding the operation of the waste collection unit 12A and the status of the surgical procedure. As one example, the controller 18A may be configured to determine a quantitative blood loss (QBL) for a surgical procedure based on the data generated by the sensor(s) 22A.

In some instances, the waste collection unit 12A may also include a local UI 24A, which may correspond to the local UI 24 discussed above in reference to FIG. 1. The local UI 24A may be in operable communication with controller 18A, which may be configured to display information relating to operation of the waste collection unit 12A based on the data generated by the sensor(s) 22A. The local UI 24A may also be configured to provide audible tones to a user, and to accept user inputs. For instance, the local UI 24A may include a touchscreen display. Surgical personnel may thus be able to enter commands to regulate the waste collection unit 12A based on the pressing of button images presented on the touchscreen display of the local UI 24A.

Referring now to FIGS. 2 and 3, the waste collection unit 12A may additionally include a reader 44A positioned adjacent each receiver 34A to allow the controller 18A of the waste collection unit 12A to communicate with a tag 36A positioned on or in the manifold 16A when the manifold 16A is inserted into the receiver 34A. In some instances, the controller 18A may be configured to inhibit activation of the vacuum pump 110 until the manifold 16A has been inserted into the receiver 34A and authenticated by the controller 18A, such as based on data read from the tag 36A via the reader 44A. The controller 18A may thus serve as a master override that prohibits the vacuum pump 110 from being actuated unless an appropriate manifold 16A is fitted to the waste collection unit 12A. If the user tries to actuate the vacuum pump 110 without a manifold 16A being inserted and authenticated, the controller 18A may be configured to prevent activation of the vacuum pump 110. The controller 18A may also cause a warning message to be presented on the local UI 24A and/or the display 28A of the user interface device 14, such as via a wireless connection established as described in more detail below.

The tag 36A may be configured to be detected by the reader 44A when the manifold 16A is in the fully inserted operative position within the receiver 34A. For example, the reader 44A may be positioned relative to the receiver 34A such that the tag 36A is only detectable by the reader 44A when the manifold 16A is in the fully inserted operative position within the receiver 34A (e.g., is in fluid communication with the corresponding waste container 106). In this way, should an article such as an incompatible manifold be incapable of being inserted into the fully inserted operative position, no data communication may be established between a tag of the article and the reader 44A, and the controller 18A may thus prevent operation of the waste collection unit 12A.

Various other features of the manifold 16A and the waste collection unit 12A are contemplated. To that end, the disclosures of International Publication No. WO 2019/222655, published Nov. 21, 2019, and U.S. Pat. No. 10,471,188 , issued Nov. 12, 2019, are each hereby incorporated by reference herein in their entirety.

Referring again to FIG. 2, the surgical sponge management device 14A may include a stand 132 and an electronics subsystem 134. The stand 132 may include a base 138 that may be wheeled so as to maneuver the surgical sponge management device 14A within a medical facility. The electronics subsystem 134 may include a controller 26A, a display 28A, an input device 30A, a wireless communication device 32A, and a reader 42A, which may respectively correspond to the controller 26, display 28, input device(s) 30, wireless communication device 32, and reader 42 discussed above in reference to FIG. 1. As shown in the illustrated example, the display 28A may be incorporated into a tablet 139 removably coupleable to the stand 132. Although the illustrated example shows the controller 26A and wireless communication devices 32 as being separate from the tablet 139, in other implementations, these components may be incorporated into the tablet 139 with the display 28A, and correspondingly may likewise be removably coupleable to the stand 132. Similarly, although the illustrated example shows the reader 42A as being incorporated into the tablet 139 with the display 28A, in other implementations, the reader 42A may be separate from the display 28A and tablet 139, and/or integrated with the stand 132.

The controller 26A may be configured to implement the functions, features, and processes of the surgical sponge management device 14A described herein. For instance, in addition to facilitating pairing with and functioning as a user interface device 14 for one or more surgical devices 12 such as the medical waste collection unit 12A, the controller 26A may be configured to manage surgical sponge use during a surgical procedure to prevent retention of surgical sponges within the patient.

To this end, the surgical sponge management device 14A may include a data reader 140 removably couplable to the stand 132 and in data communication with the controller 26A. The data reader 140 may be configured to read unique identification information from tags associated with surgical sponges, and forward such information to the controller 26A. In an exemplary implementation, the data reader 140 may be an RFID reader configured to detect RFID tags associated with the surgical sponges as described in commonly-owned International Publication No WO2021/041795, published Mar. 4, 2021, and commonly-owned International Publication No WO2021/097197, published May 20, 2021, each of which is hereby incorporated by reference in its entirety. Exemplary tags other than RFID tags are disclosed in commonly-owned International Publication No. WO2017/112051, published Jun. 29, 2017, which is hereby incorporated by reference in its entirety.

During a surgical procedure, the controller 26A may be configured to count in surgical sponges by reading the tag information from the sponges when positioned near the data reader 140, and to increment a counter of the quantity of surgical sponges being used during the surgical procedure. The counter may be displayed on the display 28A. The controller 26A may further be configured add the dry weight of each counted-in sponge to the dry weights of surgical sponges that were previously counted in to be used.

Either during or after the surgical procedure, the surgical sponges—used and unused—may be checked or counted out. To this end, the tag of each of the surgical sponges may again be positioned to be read by data reader 140. The controller 26A may be configured to responsively decrement the counter accordingly (e.g., subtract one from the previous quantity), and show on the display 28A the quantity indicative of the surgical sponges that remain counted in. After confirming on the display 28A that a surgical sponge has been counted out, the sponge may be placed in a pocket of a sponge sorter disposed from an arm 142 of the surgical sponge management device 14A, which may be coupled to a weight sensor 144 configured to generated data indicative of a weight of the sponge sorter. The weight sensor 144 may be communicatively coupled to the controller 26A, which may be configured to receive the generated weight data, and to calculate and display an estimated blood loss of the patient based on the weight indicated by the weight data and the previously determined dry weight of the counted-in sponges. Suitable construction and operation of several subsystems of the surgical sponge management device 14A are disclosed in commonly owned International Patent Application No. PCT/US2022/017664, filed on Feb. 24, 2022, and published as International Publication No. WO ______, the entire contents of which are hereby incorporated herein by reference.

The waste collection unit 12A and the surgical sponge management device 14A may be separately positionable within an operating suite. In some situations, the waste collection unit 12A and surgical sponge management device 14A may be placed at locations spaced apart by a distance that can preclude the use of near field communication or other proximity-based communications technologies to establish wireless communication therebetween. Therefore, to establish a wireless communication pathway that provides reliable communication between the waste collection unit 12A and the surgical sponge management device 14A during a surgical procedure, the waste collection unit 12A and the surgical sponge management device 14A may undergo a pairing process prior to the start of the surgical procedure. Once established, the wireless connection may enable the waste collection unit 12A to communicate data to the surgical sponge management device 14A for display to surgical personnel. In certain implementations, the surgical sponge management device 14A may also be configured to receive user input commands for the waste collection unit 12A, and to communicate those commands to the waste collection unit 12A via the wireless connection. In certain further implementations, the waste collection unit 12A may also transmit and receive data and commands from remote devices or systems through the surgical sponge management device 14A, such as remote devices or systems accessible via the Internet.

The waste collection unit 12A may be configured to be arranged in unidirectional or bidirectional wireless electronic communication with a user interface device 14, such as the surgical sponge management device 14A, using the manifold 16A. More specifically, prior to a surgical procedure, the manifold 16A may be brought into proximity to the reader 42A of the surgical sponge management device 14A, which may be configured to read data from the tag 36A. Thereafter, the manifold 16A may be inserted into a receiver 34A of the waste collection unit 12A, responsive to which the controller 18A may be configured to read data from the tag 36A via the reader 44A. Thereafter, the controllers 18A, 26A may be configured to complete a pairing process that establishes a wireless communication link between the wireless communication device 20A of the waste collection unit 12A and the wireless communication device 32A of the surgical sponge management device 14A. This process is described in more detail below.

As previously described, the tag 36A may store data indicative of a type identifier 38 and/or a unique identifier 40. Referring again to FIG. 3, such tag data may be stored on an RFID tag 126A of the tag 36A, may be encoded in a barcode 128A or other optical machine-readable data encoding of the tag 36A, or both. In some instances, the barcode 128A may encode one of the above datums, the type identifier 38 or the unique identifier 40, and the RFID tag 126A may store the other of these datums.

Accordingly, the tag 36A and the readers 42A, 44A may each employ optical and/or radio-based technology. For instance, the tag 36A may include an RFID tag 126A and the readers 42A, 44A may each include an RFID interrogator. Additionally or alternatively, the tag 36A may include a barcode 128A or other optical machine-readable data encoding, such as a QR code or the like, and the readers 42A, 44A may each include an optical scanner. In some instances, the reader 42A may employ optical or radio-based technology, and the reader 44A may employ the other of optical or radio-based technology. In this case, the tag 36A may include both an RFID tag 126A and a barcode 128A or other optical machine-readable data encoding, each storing the data for being read by the corresponding reader 42A, 44A to establish a wireless connection as described herein.

In some instances, the tag 36A, such as when it includes the RFID tag 126A, may include a writable or rewritable data field where a reader 42A, 44A, such as an RFID interrogator of the reader 44A, may be configured to write or rewrite data into the writable or rewritable data field to indicate usage of the manifold 16A to which the tag 36A is applied. Once used to establish a wireless connection as described herein and/or to facilitate performance of a surgical task such as for a specified period as described herein, the manifold 16A may be rendered inoperable to establish a further wireless connection between a waste collection unit 12A and a user interface device 14 (e.g., the surgical sponge management device 14A) and/or to facilitate further performance of a surgical task by virtue the written or rewritten data. Should the manifold 16A be thereafter used in an attempt to establish a wireless connection and/or facilitate performance of a surgical task as described herein, the information written to this field may cause the controller 18A and/or the controller 26A to determine that the manifold 16A has been used and/or is unsuitable for further use in a surgical operation. Responsive to making such a determination, the controller 18A and/or the controller 26A may be configured to deny establishing a wireless connection and/or facilitating performance of a surgical task using the manifold 16A, and/or to provide a corresponding indicator to the user in the form of an alert or error message, such as on the local UI 24A and/or the display 28A of the surgical sponge management device 14A.

Additionally or alternatively, responsive to a given manifold 16A being used to establish a wireless connection as described herein and/or facilitate performance of a surgical task such as for a specified period as described herein, the controller 18A and/or the controller 26A may be configured to upload the data read from the tag 36A of the manifold 16A to a used cartridge database. Such database may be maintained on one or more of the waste collection unit 12A, the surgical sponge management device 14A, or a remote server accessible over one or more networks such as the Internet. Should the manifold 16A be thereafter used in connection with an attempt to establish a wireless connection and/or facilitate performance of a surgical task as described herein, the controller 18A and/or the controller 26A may be configured to cross check the data read from the tag 36A of the manifold 16A against the used cartridge database to determine whether the tag 36A data is indicated by such database as used. If so, then the controller 18A and/or the controller 26A may be configured to deny establishing a wireless connection and/or facilitating performance of a surgical task using the manifold 16A, and/or to provide a corresponding indicator to the user in the form of an alert or error message, such as on the local UI 24A and/or the display 28A of the surgical sponge management device 14A.

FIG. 4 illustrates a further exemplary implementation in accordance with the surgical system 10 illustrated in FIG. 1. As shown in the illustrated example, a user interface device 14 may be realized as a surgical sponge management device 14A similar to that described above, one surgical device 12 may be realized as a waste collection unit 12A similar to that described above, and a further surgical device 12 may be realized as a flow monitoring device 12B. The flow monitoring device 12B may generally be configured to monitor the flow of waste material between the surgical site and the waste collection unit 12A. In other words, the flow monitoring device 12B may be configured to perform one or more of the monitoring functions of the waste collection unit 12A described above (e.g., QBL), but do so externally to the waste collection unit 12A. In some instances, any monitoring functions performed by the flow monitoring device 12B may be omitted from the waste collection unit 12A.

As shown in the illustrated example, the flow monitoring device 12B may include a cartridge interface 34 realized as a slot 34B dimensioned for receiving a cartridge 16 realized as a cassette 16B. The cassette 16B may be configured to form one or more of a fluid, mechanical or electrical coupling with the flow monitoring device 12B when inserted into the slot 34B. The cassette 16B may also include or form a functional component of the flow monitoring device 12B that facilitates performance of a surgical task, such as via the fluid, mechanical or electrical coupling.

For example, the cassette 16B may be configured to define or include a portion of a suction pathway that extends from the surgical site through one or more of the suction tubes 118 and one or more manifolds 16A inserted in the waste collection unit 12A, so that material suctioned from the surgical site via the waste collection unit 12A passes through the cassette 16B. The cassette 16B may also be configured to form a fluid coupling with the flow monitoring device 12B such that material passing through the cassette 16B also passes through the flow monitoring device 12B.

The flow monitoring device 12B may also include a controller 18B, which may correspond to the controller 18 discussed above in reference to FIG. 1. The controller 18B may provide for overall control of the flow monitoring device 12B. For instance, the controller 18B may be configured to analyze the flow of material through the suction tube(s) 118 by the vacuum generated by the waste collection unit 12A before reaching the manifold(s) 16A, and may be configured to generate blood concentration data from the analysis of the material flow representative of, for example, the concentration of blood present in the material flow. The controller 18B may further include or be in communication with a wireless communication device 20B of the flow monitoring device 12B. The wireless communication device 20B may correspond to the wireless communication device 20 discussed above in reference to FIG. 1.

The flow monitoring device 12B may also include one or more sensors 22B in communication with the controller 18B that are configured to generate operational data relating to use of the flow monitoring device 12B, such as to facilitate determination of blood concentration data as described above. The sensor(s) 22B may correspond to the sensor(s) 22 described above in reference to FIG. 1. For example and without limitation, the sensor(s) 22B may include a fluid measuring subsystem arranged to measure the volume of waste material suctioned through the cassette 16B. Additionally or alternatively, the sensor(s) 22B may include one or more flow rate sensors to measure a rate of flow of material passing through the cassette 16B. Additionally or alternatively, the sensor(s) 22B may include one or more blood concentration sensors for measuring a concentration of blood within the waste material passing through the cassette 16B. Exemplary measurement or monitoring devices are disclosed in commonly-owned International Application No. PCT/US2021/058891, filed Nov. 11, 2021, the entire contents of which are hereby incorporated by reference. The data generated by the sensor(s) 22B may provide valuable information to surgical personnel regarding the operation of the waste collection unit 12A and the status of the surgical procedure.

In some instances, the flow monitoring device 12B may also include a local UI 24B, which may correspond to the local UI 24 discussed above in reference to FIG. 1. The local UI 24B may be in operable communication with controller 18B, which may be configured to display information relating to operation of the flow monitoring device 12B based on the data generated by the sensor(s) 22B. The local UI 24B may also be configured to provide audible tones to a user, and to accept user inputs. For instance, the local UI 24B may include a touchscreen display. Surgical personnel may thus be able to enter commands to regulate the flow monitoring device 12B based on the pressing of button images presented on the touchscreen display of the local UI 24B.

The flow monitoring device 12B may additionally include a reader 44B positioned adjacent the slot 34B to allow the controller 18B of the flow monitoring device 12B to communicate with a tag 36B positioned on or in the cassette 16B when the cassette 16B is inserted into the slot 34B. In some instances, the controller 18B may be configured to inhibit suction from the waste collection unit 12A from reaching the surgical site until a cassette 16B has been inserted into the slot 34B and authenticated by the controller 18B, such as based on data read from the tag 36B via the reader 44B. As an example, the flow monitoring device 12B or cassette 16B may include a valve operable by the controller 18B and in a suction path established between the tube(s) 118 and the waste collection unit 12A when the cassette 16B is inserted into the slot 34B and coupled to the tube(s) 118 and waste collection unit 12A. The controller 18B may be configured to maintain the valve in a closed state when a cassette 16B is not inserted or authenticated, thus preventing suction generated by the waste collection unit 12A from reaching the surgical site. Responsive to a cassette 16B being inserted and authenticated, the controller 18B may be configured to open the valve, thereby allowing the suction to flow through the cassette 16B and flow monitoring device 12B.

The tag 36B may be configured to be detected by the reader 44B when the cassette 16B is in the fully inserted operative position within the slot 34B. For example, the reader 44B may be positioned relative to the slot 34B such that the tag 36B is only detectable by the reader 44B when the cassette 16B is in the fully inserted operative position within the slot 34B (e.g., is in fluid communication with the flow monitoring device 12B). In this way, should an article such as an incompatible cassette be incapable of being placed into the fully inserted operative position, no data communication may be established between a tag of the article and the reader 44B, and the controller 18B may thus prevent suction from being applied to the surgical site from the waste collection unit 12A.

Like the waste collection unit 12A, the flow monitoring device 12B and the surgical sponge management device 14A may be separately positionable within an operating suite. In some situations, the flow monitoring device 12B and surgical sponge management device 14A may be placed at locations spaced apart by a distance that can preclude the use of near field communication or other proximity-based communications technologies to establish wireless communication therebetween. Therefore, to establish a wireless communication pathway that provides reliable communication between the flow monitoring device 12B and the surgical sponge management device 14A during a surgical procedure, the flow monitoring device 12B and the surgical sponge management device 14A may undergo a pairing process prior to the start of the surgical procedure. Once established, the wireless connection may enable the flow monitoring device 12B to communicate data to the surgical sponge management device 14A for display to surgical personnel. In certain implementations, the surgical sponge management device 14A may also be configured to receive user input commands for the flow monitoring device 12B, and to communicate those commands to the flow monitoring device 12B via the wireless connection. In certain further implementations, the flow monitoring device 12B may also transmit and receive data and commands from remote devices or systems through the surgical sponge management device 14A, such as remote devices or systems accessible via the Internet.

The flow monitoring device 12B may be configured to be arranged in unidirectional or bidirectional wireless electronic communication with a user interface device 14, such as the surgical sponge management device 14A, using the cassette 16B. More specifically, prior to a surgical procedure, the cassette 16B may be brought into proximity to the reader 42A of the surgical sponge management device 14A, which may be configured to read data from the tag 36B. Thereafter, the cassette 16B may be inserted into the slot 34B of the flow monitoring device 12B, responsive to which the controller 18B may be configured to read data from the tag 36B via the reader 44B. Thereafter, the controllers 18B, 26A may be configured to complete a pairing process that establishes a wireless communication link between the wireless communication device 20B of the flow monitoring device 12B and the wireless communication device 32A of the surgical sponge management device 14A. This process is described in more detail below.

As previously described, the tag 36B may store data indicative of a type identifier 38 and/or a unique identifier 40. Such tag 36B data may be stored on an RFID tag 126B of the tag 36B, may be encoded in a barcode 128B or other optical machine-readable data encoding of the tag 36B, or both. In some instances, the barcode 128B may encode one of the above datums, the type identifier 38 or the unique identifier 40, and the RFID tag 126B may store the other of these datums.

Accordingly, the tag 36B and the readers 42A, 44B may each employ optical and/or radio-based technology. For instance, the tag 36B may include an RFID tag 126B and the readers 42A, 44B may each include an RFID interrogator. Additionally or alternatively, the tag 36B may include a barcode 128B or other optical machine-readable data encoding, such as a QR code or the like, and the readers 42A, 44B may each include an optical scanner. In some instances, the reader 42A may employ optical or radio-based technology, and the reader 44B may employ the other of optical or radio-based technology. In this case, the tag 36B may include both an RFID tag 126A and a barcode 128B or other optical machine-readable data encoding, each storing the data for being read by the corresponding reader 42A, 44B to establish a wireless connection as described herein.

In some instances, the tag 36B, such as when it includes the RFID tag 126B, may include a writable or rewritable data field where a reader 42A, 44B, such as an RFID interrogator of the reader 44B, may be configured to write or rewrite data into the writable or rewritable data field to indicate usage of the cassette 16B to which the tag 36B is applied. Once used to establish a wireless connection as described herein and/or to facilitate performance of a surgical task such as for a specified period as described herein, the cassette 16B may be rendered inoperable to establish a further wireless connection between a flow monitoring device 12B and a user interface device 14 (e.g., the surgical sponge management device 14A) and/or to facilitate further performance of a surgical task by virtue the written or rewritten data. Should the cassette 16B be thereafter used in an attempt to establish a wireless connection and/or facilitate performance of a surgical task as described herein, the information written to this field may cause the controller 18B and/or the controller 26A to determine that the cassette 16B has been used and/or is unsuitable for further use in a surgical operation. Responsive to making such a determination, the controller 18B and/or the controller 26A may be configured to deny establishing a wireless connection and/or facilitating performance of a surgical task using the cassette 16B, and/or to provide a corresponding indicator to the user in the form of an alert or error message, such as on the local UI 24B and/or the display 28A of the surgical sponge management device 14A.

Additionally or alternatively, responsive to a given cassette 16B being used to establish a wireless connection as described herein and/or to facilitate performance of a surgical task such as for a specified period as described herein, the controller 18B and/or the controller 26A may be configured to upload data read from the tag 36B of the cassette 16B to a used cartridge database. Such database may be maintained on one or more of the flow monitoring device 12B, the surgical sponge management device 14A, or a remote server accessible over one or more networks such as the Internet. Should the cassette 16B be thereafter used in connection with an attempt to establish a wireless connection and/or to facilitate performance of a surgical task as described herein, the controller 18B and/or the controller 26A may be configured to cross check the data read from the tag 36B of the cassette 16B against the used cartridge database to determine whether the tag 36B data is indicated by such database as used. If so, then the controller 18B and/or the controller 26A may be configured to deny establishing a wireless connection and/or facilitating performance of a surgical task using the cassette 16B, and/or to provide a corresponding indicator to the user in the form of an alert or error message, such as on the local UI 24B and/or the display 28A of the surgical sponge management device 14A.

In some instances, the flow monitoring device 12B may be configured to communicate with the user interface device 14 via a wireless connection formed between the waste collection unit 12A and the user interface device 14 using a manifold 16A as described herein. Alternatively, the waste collection unit 12A may be configured to communicate with the user interface device 14 via a wireless connection formed between the flow monitoring device 12B and the user interface device 14 using a cassette 16B as described herein. To either end, the flow monitoring device 12B may be communicatively coupled to the waste collection unit 12A, such as via a data communication cable extending therebetween. As one example, such data communication cable may be integrated with a fluid tube connecting the cassette 16B to the manifold 16A, and may function to connect the controller 18B of the flow monitoring device 12B with the controller 18A of the waste collection unit 12A. In this way, in addition to forming a fluid coupling, the cassette 16B may be configured to form an electrical coupling with the flow monitoring device 12B when inserted in the slot 34B, and/or a manifold 16A may be configured to form an electrical coupling with the waste collection unit 12A when inserted in a receiver 34A.

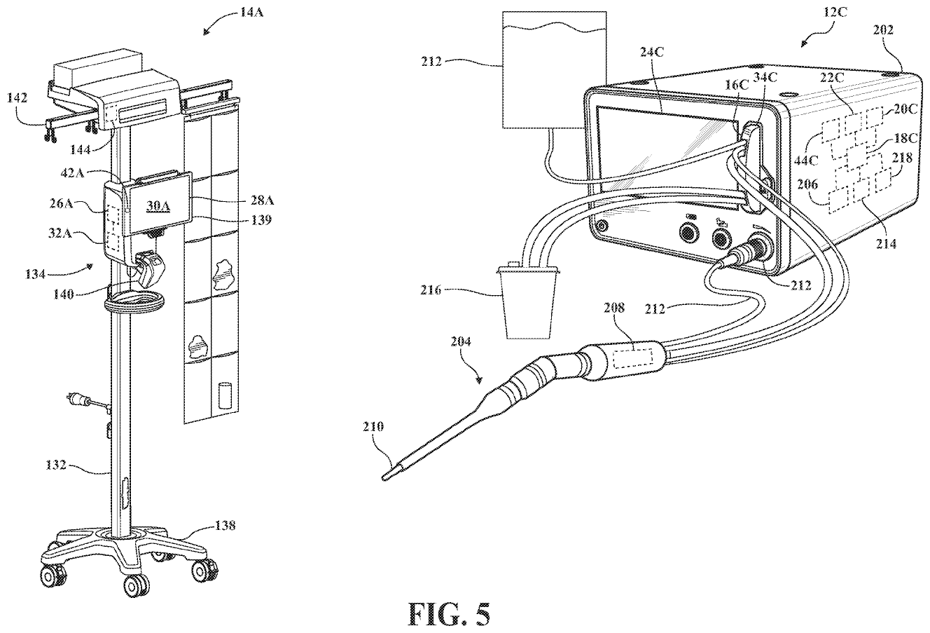

FIG. 5 illustrates another exemplary implementation in accordance with the surgical system 10 illustrated in FIG. 1. As shown in the illustrated example, the surgical device 12 may be realized as an irrigation device, or more particularly an ultrasonic aspiration and irrigation device 12C, and the user interface device 14 may be realized as the surgical sponge management device 14A described above. The ultrasonic aspiration and irrigation device 12C may be employed during a surgical procedure to ablate patient tissue, to introduce fluid into a surgical site, and to aspirate liquids and tissue from the surgical site. Introduction of an irrigating fluid may aid in the surgical procedure, such as by reducing heat generation of the surgical instruments and of the tissues adjacent the surgical site.

The ultrasonic aspiration and irrigation device 12C may include a control console 202 and an ultrasonic instrument 204. The control console 202 may be configured, such as via an integrated power supply 206, to generate and source an AC drive signal to the ultrasonic instrument 204, or more particularly to an ultrasonic transducer 208 of the ultrasonic instrument 204. Responsive to receiving the AC drive signal, the ultrasonic transducer 208 may expand and contract, causing corresponding vibrations of a tip 210 coupled to the ultrasonic transducer 208. The vibrating tip 210 may then be placed against patient tissue at a target site to cut or ablate the same.

To provide the aforementioned irrigation and aspiration at the surgical site, the control console 202 may also include a cartridge interface 34 realized as a slot 34C dimensioned for receiving a cartridge 16 realized as a cassette 16C. The cassette 16C may be configured to form one or more of a fluid, mechanical or electrical coupling with the control console 202 when inserted into the slot 34C. The cassette 16C may also include a functional component of the ultrasonic aspiration and irrigation device 12C that facilitates performance of a surgical task, such as via the fluid, mechanical or electrical coupling.

For example, the cassette 16C may define or include a portion of an irrigation pathway that extends from an irrigation fluid supply 212 through the cassette 16C and then through the ultrasonic instrument 204 to the distal region of the tip 210 for supplying fluid thereto. Upon insertion of the cassette 16C into the slot 34C, a portion of the irrigation pathway defined by or included in the cassette 16C (e.g., pump tube 213 (FIG. 6)) may engage a peristaltic pump 214 internal to the control console 202. The pump 214 may then be operated to propel the irrigating fluid through the irrigation pathway from the fluid supply 212 to the distal region of the tip 210 to introduce such fluid to the surgical site.

The cassette 16C may also define a portion of an aspiration pathway that extends from the distal region of the tip 210 through the cassette 16C and to a waste canister 216. More specifically, upon insertion of the cassette 16C into the slot 34C, a separate pathway may be formed through the cassette 16C between a vacuum source 218 internal to the control console 202 and the waste canister 216. The vacuum source 218 may then be operated to apply suction on the aspiration pathway via the separate pathway to suction fluid and other material from the surgical site into the waste canister 216.

The control console 202 may include a controller 18C configured to implement the functions, features, and processes of the control console 202 described herein. For example, the controller 18C may be coupled and configured to regulate operation of the power supply 206, pump 214, and vacuum source 218 during operation of the ultrasonic aspiration and irrigation device 12C. The controller 18C may also be in communication with a wireless communication device 20C and one or more sensors 22C. The sensor(s) 22C may be configured to generate operational data relating to operation of the ultrasonic aspiration and irrigation device 12C, and to provide information and feedback to surgical personnel based on the received data, such as on a display 28 of a remote user interface device 14 (e.g., the surgical sponge management device 14A), by communicating the information and feedback to the user interface device 14 via a wireless connection established as described in more detail below. For example and without limitation, the sensors 22C may include a voltage sensor for measuring a voltage of the AC drive signal sourced to the ultrasonic instrument 204, a current sensor for measuring a current of the AC drive signal sourced to the ultrasonic instrument 204, one or more flow rate sensors for measuring a rate of irrigation solution and/or waste flow through the cassette 16C, and/or one or more volume sensors for measuring a total volume of irrigation solution consumed and/or of waste collected in a surgical procedure.

In some instances, the cassette 16C may also include at least two electrical contacts 220 and an electrical conductor 222 extending therebetween (FIG. 6). Upon insertion of the cassette 16C into the slot 34C, the electrical contacts 220 may contact corresponding contacts of the of the control console 202, thus completing a circuit that enables the control console 202 to operate. As one example, insertion of the cassette 16C into the slot 34C may complete a circuit electrically coupling the controller 18C to other components of the control console 202 (e.g., reader 44C, sensors 22C, wireless communication device 20C, power supply 206, pump 214, vacuum source 218) to enable the controller 18C to communicate and/or regulate the same.

In some instances, the ultrasonic aspiration and irrigation device 12C may also include a local UI 24C in operable communication with controller 18A, which may be configured to display information relating to operation of the ultrasonic aspiration and irrigation device 12C based on the data generated by the sensor(s) 22C. The local UI 24C may also be configured to provide audible tones to a user, and to accept user inputs. For instance, the local UI 24C may be a touchscreen display. Surgical personnel may thus be able to enter commands to regulate the ultrasonic aspiration and irrigation device 12C based on the pressing of button images presented on the local UI 24C.

Various other features of the cassette 16C and ultrasonic aspiration and irrigation device 12C are contemplated. To that end, the disclosures of commonly owned International Publication No. WO 2015/021216 A1, published Feb. 12, 2015, and International Publication No. WO 2020/068823 A1, published Apr. 2, 2020, are each hereby incorporated by reference herein in their entirety.

Referring now to FIGS. 5 and 6, the control console 202 of the ultrasonic aspiration and irrigation device 12C may include a reader 44C positioned adjacent the slot 34C to allow the controller 18C of the ultrasonic aspiration and irrigation device 12C to communicate with a tag 36C positioned on or in the cassette 16C when the cassette 16C is inserted into the slot 34C. In some instances, the controller 18C may be configured to inhibit activation of ultrasonic aspiration and irrigation device 12C, or more particularly of the ultrasonic instrument 204, until the cassette 16C has been inserted and authenticated by controller 18C, such as based on data read from the tag 36C via the reader 44C. The controller 18C may thus serve as a master override that prohibits the ultrasonic instrument 204 from being actuated unless an appropriate cassette 16C is fitted to the ultrasonic aspiration and irrigation device 12C. If the user tries to actuate the ultrasonic instrument 204 without a cassette 16C being inserted and authenticated, the controller 18C may be configured to prevent activation of the ultrasonic instrument 204. The controller 18C may also cause a warning message to be presented on the local UI 24C and/or the display 28 of the user interface device 14, such as via a wireless connection as described in more detail below.

The tag 36C may be configured to be detected by the reader 44C when the cassette 16C is in a fully inserted operative position within the slot 34C. For example, the reader 44C may be positioned relative to the slot 34C such that the tag 36C is only detectable when the cassette 16C is in the fully inserted operative position within the slot 34C (e.g., is in fluid communication with the pump 214 and the vacuum source 218. In this way, should an article such as an incompatible cassette be incapable of being inserted into the fully inserted operative position, no data communication may be established between a tag of the article and the reader 44C, and the controller 18C may thus prevent operation of the ultrasonic instrument 204.

The ultrasonic aspiration and irrigation device 12C may be configured to be arranged in unidirectional or bidirectional wireless electronic communication with a user interface device 14, such as the surgical sponge management device 14A, using the cassette 16C. More specifically, prior to a surgical procedure, the cassette 16C may be brought into proximity to the reader 42A of the surgical sponge management device 14A, which may be configured to read the data from the tag 36C. Thereafter, the cassette 16C may be inserted into the slot 34C of the ultrasonic aspiration and irrigation device 12C, responsive to which the controller 18C may be configured to read the data from the tag 36C via the reader 44C. Thereafter, the controllers 18C, 26A may be configured to complete a pairing process that establishes a wireless communication link between the wireless communication device 20C of the ultrasonic aspiration and irrigation device 12C and the wireless communication device 32A of the surgical sponge management device 14A. This process is described in more detail below.

As previously described, the tag 36C may store data indicative of a type identifier 38 and/or a unique identifier 40. Such tag data may be stored in an RFID tag 126C of the tag 36C, may be encoded in a barcode 128C or other optical machine-readable data encoding of the tag 36C, or both. In some instances, the barcode 128C may encode one of the above datums, the type identifier 38 or the unique identifier 40, and the RFID tag 126C may store the other of these datums.

Accordingly, the tag 36C and the readers 42A, 44C may each employ optical and/or radio-based technology. For instance, the tag 36C may include an RFID tag 126C and the readers 42A, 44C may each include an RFID interrogator. Additionally or alternatively, the tag 36C may include a barcode 128C or other optical machine-readable data encoding, such as a QR code or the like, and the readers 42A, 44C may each include an optical scanner. In some instances, the reader 42A may employ optical or radio-based technology, and the reader 44C may employ the other of optical or radio-based technology. In this case, the tag 36C may include both an RFID tag 126C and a barcode 128C or other optical machine-readable data encoding, each storing the data for being read by the corresponding reader 42A, 44C to establish a wireless connection as described herein.

In some instances, the tag 36C, such as when it includes the RFID tag 126C, may include a writable or rewritable data field where a reader 42A, 44C, such as an RFID interrogator of the reader 44C, may be configured to write or rewrite data into the writable or rewritable data field to indicate usage of the cassette 16C to which the tag 36C is applied. Once used to establish a wireless connection as described herein and/or to facilitate performance of a surgical task such as for a specified period as described herein, the cassette 16C may be rendered inoperable to establish a further wireless connection between an ultrasonic aspiration and irrigation device 12C and a user interface device 14 (e.g., the surgical sponge management device 14A) and/or to facilitate further performance of a surgical task by virtue the written or rewritten data. Should the cassette 16C be thereafter used in an attempt to establish a wireless connection and/or facilitate performance of a surgical task as described herein, the information written to this field may cause the controller 18C and/or the controller 26C to determine that the cassette 16C has been used and/or is unsuitable for further use in a surgical operation. Responsive to making such a determination, the controller 18C and/or the controller 26A may be configured to deny establishing a wireless connection and/or facilitating performance of a surgical task using the cassette 16C, and/or to provide a corresponding indicator to the user in the form of an alert or error message, such as on the local UI 24B and/or the display 28A of the surgical sponge management device 14A.

Additionally or alternatively, responsive to a given cassette 16C being used to establish a wireless connection as described herein and/or to facilitate performance of a surgical task such as for a specified period as described herein, the controller 18C and/or the controller 26A may be configured to upload data read from the tag 36C of the cassette 16C to a used cartridge database. Such database may be maintained on one or more of the ultrasonic aspiration and irrigation device 12C, the surgical sponge management device 14A, or a remote server accessible over one or more networks such as the Internet. Should the cassette 16C be thereafter used in connection with an attempt to establish a wireless connection and/or to facilitate performance of a surgical task as described herein, the controller 18C and/or the controller 26A may be configured to cross check the data read from the tag 36C of the cassette 16C against the used cartridge database to determine whether the tag 36C data is indicated by such database as used. If so, then the controller 18C and/or the controller 26A may be configured to deny establishing a wireless connection and/or facilitating performance of a surgical task using the cassette 16C, and/or to provide a corresponding indicator to the user in the form of an alert or error message, such as on the local UI 24C and/or the display 28A of the surgical sponge management device 14A.

It will be appreciated that multiple surgical devices 12 may be wirelessly connected to a same user interface device 14 at the same time using the procedures described herein. For instance, referring to the examples above, surgical personnel may pair the waste collection unit 12A with the surgical sponge management device 14A via the manifold 16A as described herein, and then proceed to pair the ultrasonic aspiration and irrigation device 12C to the surgical sponge management device 14A via the cassette 16C as described herein. In this way, the user interface device 14 may act as a user interface for each surgical device 12 via its respective wireless connection, and also aggregate data from multiple surgical devices 12 so as to provide a complete picture of the status of the surgical procedure to surgical personnel.

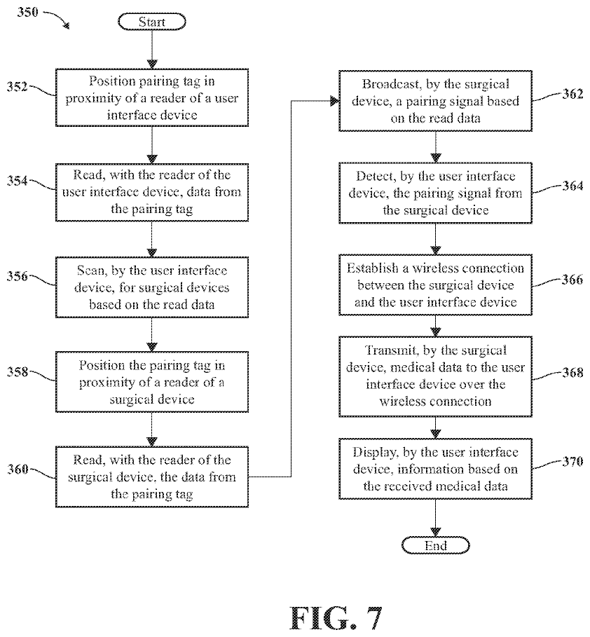

FIG. 7 illustrates a method 350 for pairing a surgical device 12 with a user interface device 14 in an operating suite to establish a wireless connection therebetween. In block 352, a pairing tag 36 may be positioned in proximity of a reader 42 of the user interface device 14. As described above, the tag 36 may be integrated with a cartridge 16 configured to be received by the surgical device 12 to form a functional component thereof (e.g., to enable performance of a surgical task by the surgical device 12).

In block 354, data may be read from the tag 36 with the reader 42 of the user interface device 14, such as by the controller 26 of the user interface device 14. The read data may include a type identifier 38 and/or a unique identifier 40. Responsive to a successful reading of such data, the controller 26 may be configured to provide a feedback response in the form of a visual or audible indication, such as via the display 28.

In block 356, surgical devices 12 may be scanned for based on the data read from the tag 36, such as by the user interface device 14. More specifically, the controller 26 of the user interface device 14 may operate the wireless communication device 32 to scan for a broadcasted pairing signal based at least on the unique identifier 40 of the tag 36. In some instances, prior to initiating the scan, the controller 26 may be configured to verify that read type identifier 38 corresponds to a cartridge 16 as described herein.

In block 358, the tag 36 may be positioned in proximity of a reader 44 of the surgical device 12. To this end, the cartridge 16 including the tag 36 may be coupled to the surgical device 12, such as by disposing the cartridge 16 within the cartridge interface 34 of the surgical device 12. The act of disposing the cartridge 16 within the cartridge interface 34 of the surgical device 12 may include forming one or more of a mechanical, fluid, or electrical coupling between the surgical device 12 and the cartridge 16, such as to provide for interaction therebetween. In some instances, the sensor(s) 22 of the surgical device 12 may include a sensor (e.g., mechanical sensor, optical sensor) for detecting the insertion of the cartridge 16 into the cartridge interface 34. In some instances, the reader 44 of the surgical device 12 may function as such sensor. For instance, responsive to the reader 44 detecting the presence of a tag 36, the controller 18 of the surgical device 12 may be configured to determine that a cartridge 16 has been inserted into the cartridge interface 34 of the surgical device 12.

Responsive to insertion of the cartridge 16, in some instances such that the cartridge 16 is in a fully inserted operative position, the tag 36 may come within range of the reader 44 of the surgical device 12. Thereafter, in block 360, the data of the tag 36 may be read with the reader 44 of the surgical device 12, such as by the controller 18 of the surgical device 12. In some instances, the controller 18 may be configured to read the tag 36 data responsive to receiving data from the sensor(s) 22 indicative of the insertion. The controller 18 may also be configured to provide a feedback response in the form of a visual or audible indication upon successfully reading the tag 36 data, such as via the local UI 24.

In block 362, a pairing signal may be broadcast based on the data read from the tag 36. More specifically, the controller 18, such as via the wireless communication device 20, may be configured to generate and broadcast a pairing signal including at least a portion of the read data, such as the unique identifier 40.

In some instances, the controller 18 and/or wireless communication device 20 may be configured to generate the pairing signal so to as incorporate both data read from the tag 36 and data specific to the surgical device 12, or more particularly specific to the wireless communication device 20. For instance, referring again to FIG. 1, the surgical device 12 may include an internal memory, such as incorporated in the wireless communication device 20 or the controller 18, storing one or more datums specific to the surgical device 12, such as a device address 224 and/or a device name 226. The device address 224 may be an address that uniquely identifies the surgical device 12 and/or wireless communication device 20 of the surgical device 12, and may be non-editable (e.g., MAC address). The device name 226 may be a name that identifies the surgical device 12, and may be editable. Responsive to receiving the tag 36 data, the controller 18 and/or wireless communication device 20 may be configured to change the device name 226 to include at least a portion of the read data, such as the unique identifier 40. The wireless communication device 20 may then be configured to broadcast the device address 224 and device name 226, including the incorporated tag 36 data, in a pairing signal.

In block 366, the pairing signal may be detected, such as via the scanning referenced in block 356 above being performed by the user interface device 14. Thereafter, in block 366, a wireless connection may be established between the surgical device 12 and the user interface device 14 based on the pairing signal and the read tag 36 data. More specifically, responsive to detecting the pairing signal such as via the wireless communication device 32, the controller 26 of the user interface device 14 may be configured to compare the device name 226 indicated by the pairing signal to the data read with the reader 42 of the user interface device 14 to determine a match. For instance, the controller 26 may be configured to determine if the unique identifier 40 indicated by the device name 226 of the pairing signal matches the unique identifier 40 previously read by the controller 26 via the reader 42 of the user interface device 14. Assuming the device name 226 of the pairing signal also indicates the type identifier 38 read from the cartridge 16, the controller 26 may also be configured to determine if the type identifier 38 indicated by the device name 226 of the pairing signal matches the type identifier 38 previously read by the controller 26 via the reader 42 of the user interface device. Responsive to one or more of the above read datums matching, the controller 26 may cause the wireless communication device 32 to establish a connection with the wireless communication device 20 of the surgical device 12, such as using the device address 224 indicated by the pairing signal.

In some examples, contemporaneously with establishing a wireless connection with a surgical device 12, the controller 26 of the user interface device 14 may be configured to execute a user interface device application in accordance with the type identifier 38 read from the tag 36. In particular and as described above, different types of cartridges 16 may have different surgical applications. For instance, different types of surgical devices 12 may each be associated with a different type of cartridge 16. Additionally, a given surgical device 12 may be configured to receive cartridges 16 of different types. As an example, the waste collection unit 12A may be configured to receive different types of manifolds 16A, such as a regular type manifold 16A and a QBL-enabled type manifold 16A. Depending on the type of cartridge 16 indicated by the read type identifier 38, the controller 26 of the user interface device 14 may be configured to execute functionality and bring up user interface components specific to the type of cartridge 16 on the display 28 of the user interface device 14. For instance, continuing with the above example, responsive to the type identifier 38 indicated a QBL-enabled type manifold 16A, the controller 26 may be configured to show QBL user interface elements on the display 28 that would not be displayed with a regular type manifold 16A.

Following establishment of the wireless connection between the surgical device 12 and the user interface device 14, in block 368, data relating to operation of the surgical device 12 in performance of a surgical task, such as data generated by the sensor(s) 22, may be transmitted over the wireless connection, such as by the controller 18 of the surgical device 12. In block 370, such data may be processed and/or displayed by the user interface device 14, such as by the controller 26 and/or on the display 28 of the user interface device 14.

Aspects described herein provide systems, methods, and computer program products for intuitively establishing reliable wireless connections between one or more surgical devices 12 and a user interface device 14, such as by leveraging a functional component that is operative with each surgical device 12 to perform a surgical task and that is removeable from the surgical device 12 and/or disposable (e.g., the cartridge 16). The examples described herein also enable surgical devices 12 to be easily connected to different user interface devices 14, such as to utilize the surgical device 12 in connection with different procedures and or in different locations, without the need for engaging in complex pairing processes or requiring limiting proximity-based protocols.

The flexibility and ease of establishing wireless connections offered by aspects present disclosure enables surgical personnel to digest data and control surgical devices 12 from a single point of access, namely, the user interface device 14, which may be conveniently positioned relative to the surgical personnel. Furthermore, pairing multiple surgical devices 12 to a single user interface device 14 using the examples described herein may provide advantages in a data reporting and analysis that is helpful to the surgical personnel. For instance, a given user interface device 14 may be paired with multiple surgical devices 12 each monitoring blood loss of the patient at different points. Such data can be transmitted wirelessly to the user interface device 14, which may then aggregate blood loss data from the multiple sources to represent the total amount of blood loss from the patient due to the surgical procedure. Combining the blood loss data from multiple sources can provide more accurate real-time data to the surgical team.

The method steps described herein with respect to generating data, transmitting data and displaying data may be performed in a continuously repeating cycle as a stream of data to provide a real-time indication, or as near to real-time as technologically feasible, to the user. The cycle frequency will be dependent on the specific sensor or measurement being implemented, on the communication protocol selected for the wireless communication devices, and the refresh rate of the display, along with the processing speed of the controllers in the surgical device and the user interface device.

As one non-limiting example, each controller 18, 26 may include a processor, memory, and non-volatile storage. The processor may include one or more devices selected from microprocessors, micro-controllers, digital signal processors, microcomputers, central processing units, field programmable gate arrays, programmable logic devices, state machines, logic circuits, analog circuits, digital circuits, and/or any other devices that manipulate signals (analog or digital) based on operational instructions read into the memory, such as from the non-volatile storage. The memory may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, and/or any other device capable of storing information. The non-volatile storage may include one or more persistent data storage devices such as a hard drive, optical drive, tape drive, non-volatile solid state device, and/or any other device capable of persistently storing information.

The processor may be configured to read into memory, such as from the storage, and operate under control of software embodied by computer-executable instructions. The computer-executable instructions may be compiled or interpreted from a variety of programming languages and/or technologies, including, without limitation, and either alone or in combination, Java, C, C++, C #, Objective C, Fortran, Pascal, Java Script, Python, Perl, and PL/SQL. The computer-executable instructions may be configured, upon execution by the processor, to cause the processor to implement the functions, features, processes, and methods of the respective controller 18, 26 described herein. In this way, the respective controller 18, 26, or more particularly the processor of the respective controller 18, 26, may be considered as being configured or programmed to implement the functions, features, processes, and methods of the surgical device 12 and user interface device 14 respectively.

In this application, including the definitions below, the term “processor” or “controller” may be replaced with the term “circuit”, and vice-versa. The term “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA), a controller circuit (shared, dedicated, or group) that executes code; a memory circuit (shared, dedicated, or group) that stores code executed by the controller circuit; other suitable hardware components that provide the described functionality; or a combination of some or all of the above, such as in a system-on-chip.

The controller may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various configurations the controller may actually communicate via a communications system. The communications system includes physical and/or virtual networking equipment such as hubs, switches, routers, and gateways. In some configurations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various configurations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 10182-2008 (commonly called “VHDL”). The hardware description language may be used to manufacture and/or program a hardware circuit. In some configurations, some or all features of a controller may be defined by a language, such as IEEE 1666-2005 (commonly called “System C”), that encompasses both code, as described below, and hardware description.

The term code, as used above, may include software, firmware, and/or microcode, and may refer to programs, routines, functions, classes, data structures, and/or objects. The term shared controller circuit encompasses a single controller circuit that executes some or all code from multiple controllers. The term group controller circuit encompasses a controller circuit that, in combination with additional controller circuits, executes some or all code from one or more controllers. References to multiple controller circuits encompass multiple controller circuits on discrete dies, multiple controller circuits on a single die, multiple cores of a single controller circuit, multiple threads of a single controller circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory device is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks and flowchart elements described above serve as software specifications, which can be translated into the computer programs by the routine work of a skilled technician or programmer.

The computer programs include controller-executable instructions that are stored on at least one non-transitory computer-readable medium. The computer programs may also include or rely on stored data. The computer programs may encompass a basic input/output system (BIOS) that interacts with the hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Precontroller), Scala, Eiffel, Smalltalk, Erlang, Ruby, Flash®, Visual Basic®, Lua, MATLAB, SENSORLINK, and Python®).

The foregoing description is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

Certain implementations may be described with reference to the following exemplary clauses:

Clause 1—A method of establishing a wireless connection between a surgical device and a user interface device via a cartridge that includes a tag storing a unique identifier, wherein the surgical device includes a vacuum source, a first reader, and a first wireless communication device, and wherein the user interface device includes a second reader, and a second wireless communication device, the method comprising the steps of: positioning the cartridge in proximity to the second reader; confirming, by output displayed on the user interface device, the unique identifier of the tag has been read by the second reader; coupling the cartridge with the surgical device to position the cartridge in proximity to the first reader, wherein the first reader is configured to read the unique identifier of the tag, and wherein the wireless connection between the surgical device and the user interface device is established based on the second wireless communication device scanning for the unique identifier read by the second reader and the first wireless communication device broadcasting the unique identifier read by the first reader; operating the vacuum source to draw surgical waste through the cartridge; and viewing, on the user interface device, information transmitted to the user interface device from the surgical device via the wireless connection.

Clause 2—The method of clause 1, wherein the surgical device includes a receiver, and wherein the step of coupling the cartridge with the surgical device further comprises inserting the cartridge into the receiver to establish a mechanical connection and position the cartridge in proximity to the first reader.

Clause 3—The method of clause 1 or 2, further comprising confirming, by output displayed on the user interface device, the unique identifier of the tag has been read by the first reader.

Clause 4—The method of any one of clauses 1-3, further comprising confirming, by output displayed on the user interface device, the wireless connection has been established.

Clause 5—The method of any one of clauses 1-4, further comprising: positioning the surgical device within an operating suite at a first location; and positioning the user interface device within the operating suite at a second location that precludes use of near field communication to establish the wireless connection.

Clause 6—A method of establishing a wireless connection between a surgical device and a user interface device via a cartridge for being removably received by the surgical device and that includes a tag storing a unique identifier, wherein the surgical device includes a first reader and a first wireless communication device, and the user interface device includes a second reader and a second wireless communication device, the method comprising the steps of: reading, with the first reader, the unique identifier of the tag of the cartridge positioned in proximity to the first reader upon the cartridge being inserted into the surgical device, wherein the insertion of the cartridge into the surgical device results in the cartridge being positioned in proximity to the first reader, and the cartridge is configured to operate as a functional component of the surgical device during operation of the surgical device to perform a surgical task in relation to a surgical procedure; broadcasting, with the first wireless communication device, the unique identifier read with the first reader and a wireless device address of the surgical device; receiving, by the first wireless communication device, a communication from the second wireless communication device that is responsive to the second reader reading the unique identifier of the tag of the cartridge when positioned in proximity to the second reader and to the second wireless communication device receiving the broadcast including the unique identifier; and establishing the wireless connection between the surgical device and the user interface device based on the communication.

Clause 7—A method of establishing a wireless connection between a surgical device and a user interface device via a cartridge for being removably received by the surgical device and that includes a tag storing a unique identifier, wherein the surgical device includes a first reader and a first wireless communication device, the cartridge is configured to be inserted into the surgical device such that the cartridge when inserted is positioned in proximity to the first reader and is configured to operate as a functional component of the surgical device during operation of the surgical device to perform a surgical task in relation to a surgical procedure, and the user interface device includes a second reader and a second wireless communication device, the method comprising the steps of: reading, with the second reader, the unique identifier of the tag of the cartridge when positioned in proximity to the second reader; scanning, with the second wireless communication device, for the unique identifier read by the second reader; identifying, with the second wireless communications device, a broadcast from the first wireless communication device that includes the unique identifier read with the first reader and a wireless address of the surgical device; and pairing with the surgical device via the wireless device address of the broadcast.

Clause 8—A computer program product comprising instructions stored on at least one non-transitory computer readable medium and configured upon execution by at least one processor to execute the method of clause 6 or 7.

Clause 9—A system including at least one processor and at least one memory storing instructions that, upon execution by the at least one processor, cause the at least one processor to execute the method of clause 6 or 7.

1. A method of establishing a wireless connection between a surgical device and a user interface device via a cartridge for being removably received by the surgical device and that includes a tag storing a unique identifier, wherein the surgical device includes a first reader and a first wireless communication device, and the user interface device includes a second reader and a second wireless communication device, the method comprising the steps of:
reading, with the second reader, the unique identifier of the tag of the cartridge when positioned in proximity to the second reader;
reading, with the first reader, the unique identifier of the tag of the cartridge positioned in proximity to the first reader upon the cartridge being inserted into the surgical device, wherein the insertion of the cartridge into the surgical device results in the cartridge being positioned in proximity to the first reader, and the cartridge operates as a functional component of the surgical device during operation of the surgical device to perform a surgical task in relation to a surgical procedure;
broadcasting, with the first wireless communication device, the unique identifier read with the first reader and a wireless device address of the surgical device;
scanning, with the second wireless communication device, for the unique identifier read by the second reader; and
pairing the surgical device and the user interface device via the wireless device address based on the scanned unique identifier matching the broadcasted unique identifier so as to establish the wireless connection between the surgical device and the user interface device.

2. The method of claim 1, comprising:
generating, by the surgical device, a wireless device name for the surgical device including the unique identifier; and
broadcasting, with the first wireless communication device, the wireless device name.

3. The method of claim comprising:
detecting, by the surgical device, the insertion of the cartridge within the surgical device; and
reading, with the first reader, the unique identifier of the tag based on the detected insertion.

4. (canceled)

5. The method of claim 1, wherein the first and second readers are optical sensors, and the tag is a machine-readable optical code.

6.-7. (canceled)

8. The method of claim 1, wherein the surgical device includes a vacuum source in fluid communication with the receiver, and the cartridge is a manifold configured to be removably coupled with a suction tube, the method comprising operating the vacuum source of the surgical device to draw surgical waste through the suction tube and the manifold such that the manifold facilitates the wireless connection and subsequently operates as the functional component of the surgical device during operation of the surgical device to draw surgical waste.

9. The method of claim 8, wherein the surgical device comprises a waste container in fluid communication with the receiver and a fluid measuring device operably coupled to the waste container, and the method comprising:
determining, with the fluid measuring device, fluid volume data as a measure of fluid within the waste container; and
transmitting the fluid volume data from the surgical device to the user interface device via the wireless connection for display.

10. The method of claim 9, wherein one of the surgical device and the cartridge includes a sensor for determining a concentration of blood within the surgical waste, the method comprising:
determining, with the sensor, blood concentration data as a measure of the concentration of blood within the surgical waste;
determining blood volume data as a measure of a blood volume within the waste container based on the blood concentration data and the fluid volume data; and
transmitting the blood volume data from the surgical device to the user interface device via the wireless connection for display.

11. (canceled)

12. The method of claim 9, wherein the determining and the transmitting steps are performed continuously for the user interface device to update the displayed data in real-time.

13. The method of claim 1, wherein the tag stores data indicating a type of the cartridge, and the method comprising:
reading, with the second reader, the cartridge type data of the tag of the cartridge when positioned in proximity to the second reader; and
responsive to establishing the wireless connection with the surgical device, displaying, by the user interface device, one or more user interface elements associated with the cartridge type.

14. A system for use within an operating suite, the system comprising:
a cartridge that includes a tag storing a unique identifier;
a surgical device comprising a receiver, a first reader coupled to the receiver, and a first wireless communication device, wherein the receiver is configured to removably receive the cartridge so that the tag is positioned in proximity with the first reader; and
a user interface device positionable remote from the surgical device, the user interface device comprising a display, a second reader, and a second wireless communication device,
wherein the first wireless communication device is configured to broadcast the unique identifier and a wireless device address of the surgical device based on a reading of the tag by the first reader, and wherein the second wireless communication device is configured to scan for the unique identifier based on a reading of the tag by the second reader so as to pair and establish a wireless connection between the surgical device and the user interface device via the wireless device address.

15.-24. (canceled)

25. A surgical device for establishing a wireless connection with a user interface device via a removable cartridge including a tag storing a unique identifier, the surgical device comprising:
a wireless communication device;
a reader;
a receiver configured to removably receive the cartridge such that when the cartridge is inserted into the receiver the tag is positioned in proximity with the reader and the cartridge is configured to operate as a functional component of the surgical device to perform a surgical task in relation to a surgical procedure; and
at least one processor coupled to the reader and the wireless communication device and configured to:
reading, with the reader, the unique identifier of the tag of the cartridge positioned in proximity to the reader upon the cartridge being inserted into the receiver of the surgical device,
broadcast, with the wireless communication device, the unique identifier read with the reader and a wireless device address of the surgical device; and
establish, with the wireless communication device, the wireless connection between the surgical device and the user interface device based on a communication from the user interface device that is responsive to the broadcast of the unique identifier and wireless device address of the surgical device.

26. The surgical device of claim 25, wherein the at least one processor is configured to:
generate a wireless device name for the surgical device including the unique identifier; and
broadcast, with the wireless communication device, the wireless device name.

27. The surgical device of claim 25, wherein the at least one processor is configured to:
detect the insertion of the cartridge within the surgical device; and
read, with the reader, the unique identifier of the tag based on the detected insertion.

28.-29. (canceled)

30. The surgical device of claim 25, comprising a cartridge interface configured to form one or more of a mechanical, electrical, or fluid coupling relating to the surgical task with the cartridge upon being inserted into the receiver.

31. The surgical device of claim 30, comprising a cartridge interface configured to establish the mechanical coupling relating to the surgical task with the cartridge upon being inserted into the receiver.

32. The surgical device of claim 25, comprising a vacuum source in fluid communication with the receiver, wherein the cartridge is a manifold configured to be removably coupled with a suction tube, and the vacuum source is configured to draw surgical waste through the suction tube and the manifold such that the manifold facilitates the wireless connection and subsequently operates as the functional component of the surgical device during operation of the surgical device to draw surgical waste.

33. The surgical device of claim 32, comprising a waste container in fluid communication with the receiver and a fluid measuring device operably coupled to the waste container, wherein the at least one processor is configured to:
determine, with the fluid measuring device, fluid volume data as a measure of fluid within the waste container; and
transmit the fluid volume data from the surgical device to the user interface device via the wireless connection for display.

34. The surgical device of claim 33, comprising a sensor for determining a concentration of blood within the surgical waste, wherein the at least one processor is configured to:
determine, with the sensor, blood concentration data as a measure of the concentration of blood within the surgical waste;
determine blood volume data as a measure of a blood volume within the waste container based on the blood concentration data and the fluid volume data; and
transmit the blood volume data from the surgical device to the user interface device via the wireless connection for display.

35. The surgical device of claim 25, comprising a waste container, a vacuum source in fluid communication with the waste container, a first sensor for monitoring a flow rate of surgical waste, and a second sensor for determining a concentration of blood within the surgical waste, wherein the at least one processor is configured to:
operate the vacuum source of the surgical device to draw surgical waste through the cartridge;
determine, with the second sensor, a concentration of blood within the surgical waste being drawn through the cartridge;
determine, with the first sensor, a flow rate of the surgical waste being drawn through the cartridge;
determine blood volume data as a measure of blood volume within the waste container based on the determined concentration of blood and the determined flow rate; and
transmit the blood volume data from the surgical device to the user interface device with the wireless connection for display.

36. The surgical device of claim 33, wherein the at least one processor is configured to perform the determinations and transmissions continuously for the user interface device to update the displayed data in real-time.

37.-38. (canceled)
